(19) United States
(12) Patent Application Publication
(10) Pub. No.: US 2021/0298818 A1
(43) Pub. Date: Sep. 30, 2021

(54) ELECTROSURGICAL INSTRUMENT AND SURGICAL SYSTEM

(71) Applicant: MEDICAROID CORPORATION, Kobe-shi (JP)

(72) Inventor: Kaoru TAKAHASHI, Kobe-shi (JP)

(73) Assignee: MEDICAROID CORPORATION, Kobe-shi (JP)

(21) Appl. No.: 17/210,349

(22) Filed: Mar. 23, 2021

(30) Foreign Application Priority Data
Mar. 25, 2020 (JP) 2020-055008

(51) Int. Cl. A61B 18/14; A61B 34/30

(52) U.S. Cl. CPC A61B 18/14; A61B 2018/00178; A61B 34/30

(57) ABSTRACT

An electrosurgical instrument according to an aspect may include a housing and an electric connection part provided to the housing to be electrically connected to a power supply. The electric connection part includes: an electrode to be electrically connected to the power supply; a plurality of divided members configured to hold the electrode therebetween; and a coupling member that couples the plurality of divided members.

### CROSS REFERENCE TO RELATED APPLICATIONS

This application claims priority to Japanese Patent Application No. 2020-055008 filed on Mar. 25, 2020, the entire contents of which are incorporated herein by reference.

### BACKGROUND ART

The disclosure may relate to an electrosurgical instrument that includes an electric connection part including an electrode to be electrically connected to an electric power supply, and a surgical system that includes the electrosurgical instrument.

In a related art, there has been known an electrosurgical instrument that includes an electric connection part including an electrode to be electrically connected to an electric power supply.

U.S. Pat. No. 9,526,560 discloses an electrosurgical instrument that includes an electric connection part including a banana plug (an electrode) to be electrically connected to an electric power supply. The electric connection part includes a banana plug holding member (an electrode holder) that holds the banana plug.

In U.S. Pat. No. 9,526,560, the banana plug is assembled by being inserted into the banana plug holding member from one direction, and a connector of a cable connected to a power supply is connected to the banana plug.

### SUMMARY

Such an electrosurgical instrument in which the electrode is inserted and held, such as being disclosed in U.S. Pat. No. 9,526,560, is assembled by press-fitting an electrode into an electrode holder in general. In order to press-fit the electrode, a special jig is required, which may cause a problem that the assembly work of the electrosurgical instrument becomes complicated.

An object of an embodiment may be to provide an electrosurgical instrument and a surgical system that includes the electrosurgical instrument capable of simplifying the assembly work of the electrosurgical instrument.

A first aspect of the disclosure may be an electrosurgical instrument. The electrosurgical instrument includes: a housing to be attached to a robot arm; a shaft including one end and the other end, wherein a side of the one end of the shaft is connected to the housing; an end effector provided on a side of the other end of the shaft; and an electric connection part provided to the housing to be electrically connected to a power supply. The electric connection part includes: an electrode to be electrically connected to the power supply; a plurality of divided members configured to hold the electrode therebetween; and a coupling member that couples the plurality of divided members.

As described above, the electrosurgical instrument according to the first aspect of the disclosure is provided with the divided members that hold the electrode therebetween and the coupling member that couples the divided members. Accordingly, simply by coupling the plurality of divided members with the coupling member in a state where the electrode is held in the plurality of divided members, the electric connection part which holds the electrode can be assembled. Therefore, the assembly work of the electrosurgical instrument can be simplified.

A second aspect of the disclosure may be an electrosurgical instrument that includes: a base to be attached to a robot arm; a lid portion that covers the base and includes a notch; a shaft including one end and the other end, wherein a side of the one end of the shaft is connected to the base; an end effector provided on a side of the other end of the shaft; and an electric connecting part provided at the notch of the lid portion to be electrically connected to a power supply. The electric connection part includes an electrode to be electrically connected to the power supply, divided members that hold the electrode therebetween, and a coupling member that couples the divided members.

A third aspect of the disclosure may be a surgical system that includes: a patient-side apparatus includes robot arms to which an endoscope and an electrosurgical instrument; and an image processing apparatus including a power supply and configured to process an image captured by the endoscope. The electrosurgical instrument includes: a housing that is attached to one of the robot arms; a shaft including one end and the other end, wherein a side of the one end of the shaft is connected to the housing; an end effector provided on a side of the other end of the shaft; and an electric connection part provided to the housing and electrically connected to a power supply. The electric connection part includes: an electrode electrically connected to the power supply; a plurality of divided members configured to hold the electrode therebetween; and a coupling member that couples the plurality of divided members.

### BRIEF DESCRIPTION OF THE DRAWINGS

FIG. 1 is a diagram illustrating an overview of a robotic surgical system according to an embodiment.

FIG. 2 is a block diagram illustrating a view of a control-related configuration of the robotic surgical system according to an embodiment.

FIG. 3 is a diagram illustrating a perspective view of a state where an electrosurgical instrument is attached to a robot arm through an adaptor according to an embodiment.

FIG. 4 is a diagram illustrating a perspective view of a state where a lid portion and an electric connection part are detached from a base according to an embodiment.

FIG. 5 is a diagram illustrating an exploded perspective view of the electric connection part of the electrosurgical instrument according to an embodiment as seen from the Z1 direction.

FIG. 6 is a diagram illustrating an exploded perspective view of the electric connection part of the electrosurgical instrument according to an embodiment as seen from the Z2 direction.

FIG. 7 is a diagram illustrating a cross-sectional view taken along the 101-101 line in FIG. 3.

FIG. 8 is a diagram illustrating a perspective view of a state before a connecter is inserted into the electrosurgical instrument according to an embodiment.

FIG. 9 is a diagram illustrating a perspective view of a state where the connecter is inserted in the electrosurgical instrument according to an embodiment.

FIG. 10 is a diagram illustrating a view of a state where the lid portion and a second divided member of the electrosurgical instrument according to an embodiment are detached, as seen in the Z1 direction.

FIG. 11 is a diagram illustrating a cross sectional view of the electrosurgical instrument taken along the XZ plane according to an embodiment.

### DETAILED DESCRIPTION

Descriptions are provided hereinbelow for one or more embodiments based on the drawings. In the respective drawings referenced herein, the same constituents are designated by the same reference numerals and duplicate explanation concerning the same constituents is omitted. All of the drawings are provided to illustrate the respective examples only.

### (Configuration of Robotic Surgical System)

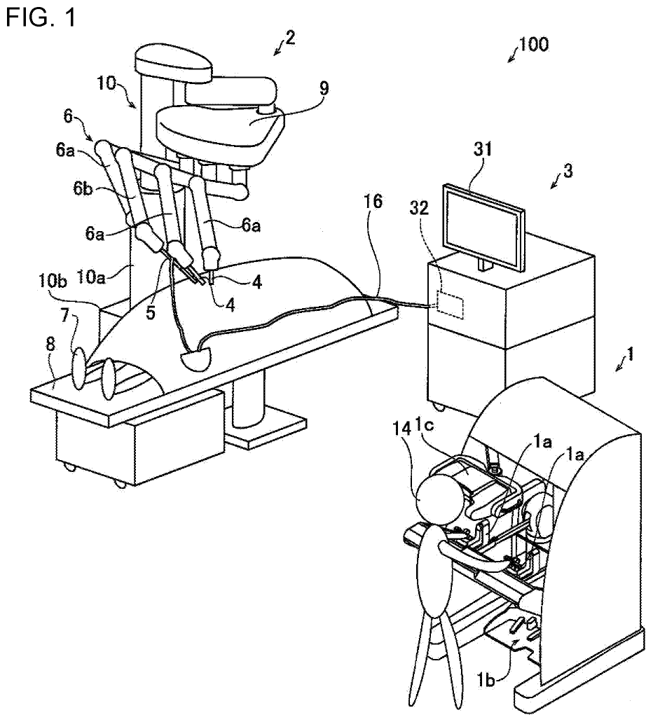

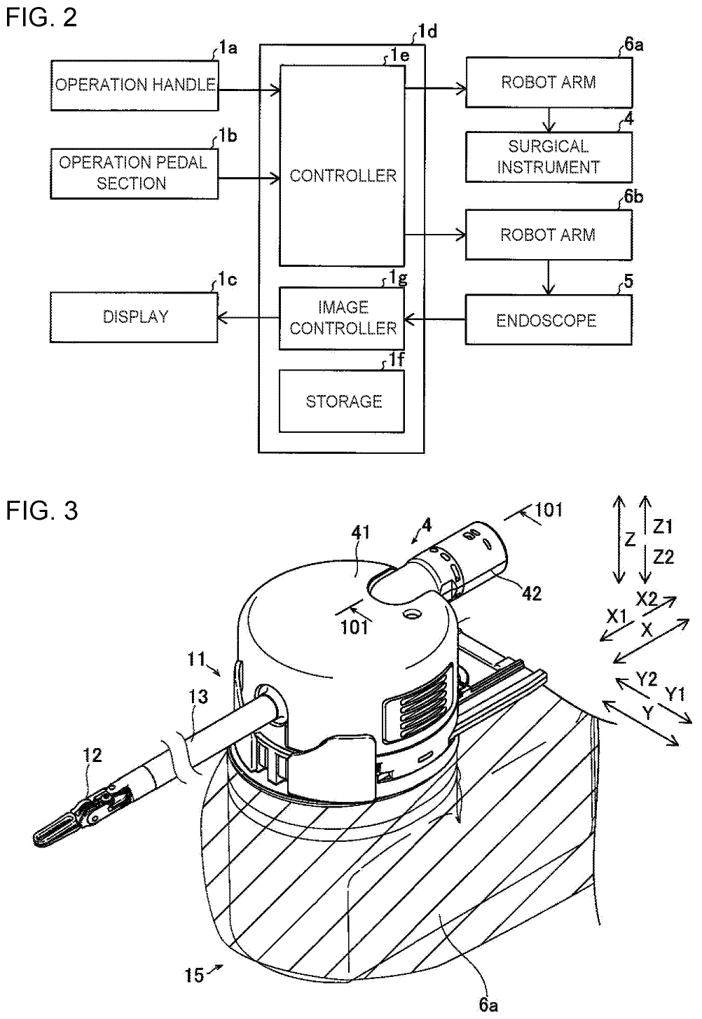

A configuration of a robotic surgical system 100 including electrosurgical instruments 4 according to an embodiment is described with reference to FIGS. 1 and 2.

As illustrated in FIG. 1, the robotic surgical system 100 includes a remote control apparatus 1, a patient-side apparatus 2, and an image processing apparatus 3. The remote control apparatus 1 is provided to remotely control medical equipment provided for the patient-side apparatus 2. When an operator 14, as a surgeon, inputs an action mode instruction to be executed by the patient-side apparatus 2, to the remote control apparatus 1, the remote control apparatus 1 transmits the action mode instruction to the patient-side apparatus 2 through a controller. In response to the action mode instruction transmitted from the remote control apparatus 1, the patient-side apparatus 2 operates medical equipment, including the electrosurgical instruments 4 attached to robot arms 6a and an endoscope 5 attached to a robot arm 6b. This allows for minimally invasive surgery.

The patient-side apparatus 2 constitutes an interface to perform a surgery for a patient 7. The patient-side apparatus 2 is positioned beside an operation table 8 on which the patient 7 is laid. The patient-side apparatus 2 includes plural robot arms 6. One 6b of the robot arms 6 holds the endoscope 5 while the others 6a of the robot arms 6 hold the electrosurgical instruments 4. The robot arms 6 are commonly supported by a platform 9. Each of the plural robot arms 6 includes plural joints. Each joint includes a driver provided with a servo-motor and a position detector such as an encoder. The robot arms 6 are configured so that the medical equipment attached to each robot arm 6 is controlled by a driving signal given through the controller and performs a desired movement.

The platform 9 is supported by a positioner 10 placed on the floor of an operation room. The positioner 10 is connected to a base 10b through a column 10a. The column 10a includes an elevating shaft adjustable in the vertical direction. The base 10b includes wheels and is movable on the floor surface.

The electrosurgical instruments 4 as the medical equipment is detachably attached to the distal ends of the robot arms 6a. The electrosurgical instruments 4 are detachably connected to the robot arms 6a of the robotic surgical system 100 through adaptors 11, as illustrated in FIG. 3. Each of the electrosurgical instrument 4 includes an end effector 12, and an elongate cylindrical tubular shaft 13, wherein the end effector 12 is provided at one end of the shaft 13. The end effector 12 is grasping forceps, scissors, a hook, a high-frequency knife, a snare wire, a clamp, or a stapler, for example. The end effector 12 is not limited to those and can be various types of treatment tools. In surgeries using the patient-side apparatus 2, the robot arms 6a introduce the electrosurgical instruments 4 into the body of the patient 7 through a cannula (trocar) placed on the body surface of the patient 7. The end effectors 12 of the electrosurgical instruments 4 are then located near a surgery site.

As illustrated in FIG. 1, to the distal end of the robot arm 6b, the endoscope 5 as the medical equipment is detachably attached. The endoscope 5 is configured to capture an image in a body cavity of the patient 7. The captured image is outputted to the remote control apparatus 1. The endoscope 5 is a 3D endoscope capable of capturing a three-dimensional image or a 2D endoscope. In surgeries using the patient-side apparatus 2, the robot arm 6b introduces the endoscope 5 into the body of the patient 7 through a trocar placed on the body surface of the patient 7. The endoscope 5 is then located near the surgery site.

The remote control apparatus 1 constitutes the interface with the operator 14. The remote control apparatus 1 is an apparatus that allows the operator 14 to operate medical equipment attached to the robot arms 6. Specifically, the remote control apparatus 1 is configured to transmit action mode instructions which are inputted by the operator 14 and are to be executed by the electrosurgical instruments 4 and the endoscope 5, to the patient-side apparatus 2 through the controller. The remote control apparatus 1 is installed beside the operation table 8 so that the operator 14 can see the condition of the patient 7 very well while operating the remote control apparatus 1 as a master apparatus, for example. The remote control apparatus 1 may be configured to transmit action mode instructions wirelessly and be installed in a room different from the operation room where the operation table 8 is installed.

The action modes to be executed by the electrosurgical instruments 4 include modes of actions to be taken by each electrosurgical instrument 4 (a series of positions and postures) and actions to be executed by the function of each electrosurgical instrument 4. When the electrosurgical instrument 4 is a pair of grasping forceps, for example, the action modes to be executed by the electrosurgical instrument 4 include roll and pitch positions of the wrist of the end effectors 12 and actions to open and close the jaws. When the electrosurgical instrument 4 is a high-frequency knife, the action modes to be executed by the electrosurgical instrument 4 include vibration of the high-frequency knife, specifically, supply of current to the high-frequency knife. When the electrosurgical instrument 4 is a snare wire, the action modes to be executed by the electrosurgical instrument 4 include a capturing action and an action to release the captured object. Further the action modes may include an action to supply current to a bipolar or monopolar instrument to burn off the surgery site.

The action mode to be executed by the endoscope 5 includes setting of the position and posture of the tip of the endoscope 5 or setting of the zoom magnification of the endoscope 5, for example.

As illustrated in FIGS. 1 and 2, the remote control apparatus 1 includes operation handles 1a, an operation pedal section 1b, a display 1c (or a display device), and a control apparatus 1d.

The operation handles 1a are provided in order to remotely operate medical equipment attached to the robot arms 6. Specifically, the operation handles 1a accept operations by the operator 14 for operating the medical equipment (the electrosurgical instruments 4 and the endoscope 5). The operation handles 1a include two operation handles 1a arranged side by side in the horizontal direction. That is, one of the two operation handles 1a is operated by the right hand of the operator 14 while the other of the two operation handles 1a is operated by the left hand of the operator 14.

The operation handles 1a extend from the rear side of the remote control apparatus 1 toward the front side. The operation handles 1a are configured to move in a predetermined three-dimensional operation region. Specifically, the operation handles 1a are configured so as to move up and down, right and left, and forward and rearward.

The remote control apparatus 1 and the patient-side apparatus 2 constitute a master-slave system in terms of controlling movements of the robot arms 6a and the robot arm 6b. That is, the operation handles 1a constitute operating parts on the master side in the master-slave system, and the robot arms 6a and 6b holding the medical equipment constitute moving parts on the slave side. When the operator 14 operates the operation handles 1a, the movement of the robot arm 6a or 6b is controlled so that the distal end portion (the end effector 12 of the electrosurgical instrument 4) of the robot arm 6a or the distal end portion (the endoscope 5) of the robot arm 6b moves following the movement of the operation handles 1a.

The patient-side apparatus 2 controls the movements of the robot arms 6a and the robot arm 6b in accordance with the set motion scaling ratio. When the motion scaling ratio is set to 1/2, for example, the end effectors 12 of the electrosurgical instruments 4 move 1/2 of the movement distance of the operation handles 1a. This allows for precise fine surgery.

The operation pedal section 1b includes plural pedals to execute medical equipment-related functions. The plural pedals include a coagulation pedal, a cutting pedal, a camera pedal, and a clutch pedal. The plural pedals are operated by a foot of the operator 14.

The coagulation pedal enables the electrosurgical instrument 4 to coagulate the surgery site. Specifically, when the coagulation pedal is operated, voltage for coagulation is applied to the electrosurgical instrument 4 to coagulate the surgery site. The cutting pedal enables the electrosurgical instrument 4 to cut the surgery site. Specifically, the cutting pedal is operated to apply voltage for cutting to the electrosurgical instrument 4 and cut the surgery site.

The camera pedal is used to control the position and orientation of the endoscope 5 that captures images within the body cavity. Specifically, the camera pedal enables control of the endoscope 5 by the operation handle 1a. That is, the position and orientation of the endoscope 5 are controllable by the operation handles 1a while the camera pedal is being pressed. The endoscope 5 is controlled by using both of the right and left operation handles 1a, for example. Specifically, when the operator 14 rotates the right and left operation handles 1a about the middle point between the right and left operation handles 1a, the endoscope 5 is rotated. When the operator 14 presses the right and left operation handles 1a together, the endoscope 5 goes further into the body cavity. When the operator 14 pulls the right and left operation handles 1a together, the endoscope 5 retracts. When the operator 14 moves the right and left operation handles 1a together up, down, right, and left, the endoscope 5 moves up, down, right, and left, respectively.

The clutch pedal is used to temporarily disconnect operation-related connection between the operation handles 1a and the robot arms 6a to stop movement of the electrosurgical instruments 4. Specifically, when the clutch pedal is being pressed, the robot arms 6a of the patient-side apparatus 2 do not work even if the operation handles 1a are operated. For example, when the operation handles 1a are operated and moved to the edge of the range of movement, the operator 14 operates the clutch pedal to temporarily disconnect the operation-related connection and then returns the operation handles 1a to the center of the range of movement. When the operator 14 stops operating the clutch pedal, the operation handles 1a are again connected to the robot arms 6a so that the operator 14 can restart the operation for the operation handles 1a around the center thereof.

The display 1c is configured to display images captured by the endoscope 5. The display 1c is composed of a scope type display or a non-scope type display. The scope type display 1c is a display configured in such a manner that the operator 14 looks into the display. The non-scope type display is a display like an open-type display that includes a flat screen and the operator 14 is able to see without looking into, such as normal displays for personal computers.

When the scope type display is attached, the scope type display displays 3D images captured by the endoscope 5 attached to the robot arm 6b of the patient-side apparatus 2. When the non-scope type display section is attached, the non-scope type display section also displays 3D images captured by the endoscope 5 provided for the patient-side apparatus 2. The non-scope type display section may display 2D images captured by the endoscope 5 provided for the patient-side apparatus.

As illustrated in FIG. 2, the control apparatus 1d includes a controller 1e, a storage 1f, and an image controller 1g, for example. The controller 1e (or a control unit) includes an arithmetic unit such as a CPU. The storage 1f includes a memory, such as a ROM and a RAM. The control apparatus 1d may be composed of a single control apparatus 1d performing centralized control or may be composed of plural control apparatuses 1d that perform decentralized control in cooperation with each other. The controller 1e determines whether an action mode instruction inputted by the operation handles 1a is to be executed by the robot arms 6a or to be executed by the endoscope 5, depending on the state of the operation pedal section 1b. When determining that the action mode instruction inputted by the operation handles 1a is to be executed by any one of the electrosurgical instruments 4, the controller 1e transmits the action mode instruction to the corresponding robot arm 6a. The robot arm 6a is thereby driven for controlling movement of the electrosurgical instrument 4 attached to the robot arm 6a.

When determining that the action mode instruction inputted by the operation handles 1a is to be executed by the endoscope 5, the controller 1e transmits the action mode instruction to the robot arm 6b. The robot arm 6b is thereby driven for control of movement of the endoscope 5 attached to the robot arm 6b.

The storage 1f stores control programs corresponding to the types of the electrosurgical instruments 4, for example. The controller 1e reads the stored control programs according to the types of the attached electrosurgical instruments 4. The action mode instructions from the operation handles 1a and/or the operation pedal section 1b of the remote control apparatus 1 thereby cause the respective electrosurgical instruments 4 to perform proper motions.

The image controller 1g transmits an image captured by the endoscope 5 to the display 1c. The image controller 1g performs processing and correcting the image when needed.

As illustrated in FIG. 1, the image processing apparatus 3 is configured to transmit the image obtained from the endoscope 5 to the remote control apparatus 1 and display the image obtained from the endoscope 5. The image processing apparatus 3 performs processing and correcting the image obtained from the endoscope 5 when needed. Specifically, the image processing apparatus 3 includes an external monitor 31. The external monitor 31 is configured to be display the image captured by the endoscope 5. The external monitor 31 is an open-type display section that includes a flat screen, such as normal displays for personal computers.

The image processing apparatus 3 includes a power supply (for example, generator) 32. The power supply 32 is configured to supply electric power to bipolar electrosurgical instruments and monopolar electrosurgical instruments. The power supply 32 and the electrosurgical instrument 4 are connected by a cable 16 provided with connectors (connection terminals) 17 (see FIG. 8) at both ends of the cable 16. With this, the electric power from the power supply 32 is supplied to the end effector 12 of the electrosurgical instrument 4.

With reference to FIGS. 3 to 11, the configuration of the electrosurgical instrument 4 according to an embodiment of the disclosure is described.

As illustrated in FIG. 3, the electrosurgical instrument 4 is an electrosurgical instrument that is detachably attached to the robot arm 6a of the robotic surgical system 100. The robot arm 6a is used in a clean area and is covered with a drape 15. In operation rooms, clean technique is used in order to prevent surgical incision sites and the medical equipment from being contaminated by pathogen, foreign matters, or the like. The clean technique defines a clean area and a contaminated area, which is other than the clean area. The surgery sites are located in the clean area. Members of the surgical team, including the operator 14, make sure that only sterile objects are placed in the clean area during surgery and perform sterilization for an object which is to be moved to the clean area from the contaminated area. Similarly, when the members of the surgical team including the operator 14 place their hands in the contaminated area, the members sterilize their hands before directly touching objects located in the clean area. Instruments used in the clean area are sterilized or are covered with a sterile drape 15.

The drape 15 is arranged between the robot arm 6a and the electrosurgical instrument 4. Specifically, the drape 15 is arranged between the adaptor 11 and the robot arm 6a. The adaptor 11 is attached to the robot arm 6a while putting the drape 15 between the adaptor 11 and the robot arm 6a. Specifically, the adaptor 11 is a drape adaptor that puts the drape 15 between the adaptor 11 and the robot arm 6a. The drape 15 is thus able to be mounted through the adaptor 11. The electrosurgical instrument 4 is attached to the adaptor 11 that is attached to the robot arm 6a with the drape 15 interposed therebetween. The robot arm 6a transmits driving force to the electrosurgical instrument 4 through the adaptor 11 to drive the end effector 12 of the electrosurgical instrument 4.

Here, the direction in which the electrosurgical instrument 4 and the adaptor 11 are adjacent to each other is referred to as a Z direction (Z axis), the electrosurgical instrument 4 side in the Z direction is referred to as a Z1 direction (Z1 side), and the opposite side (the adaptor 11 side) in the Z1 direction is referred to as a Z2 direction (Z2 side). The direction in which the shaft 13 extends is referred to as an X direction (X axis), the direction in which a connector 17 (see FIG. 8) electrically connected to the electrosurgical instrument 4 is inserted into the electrosurgical instrument 4 is referred to an X1 direction (X1 side), and the direction opposite to the X1 direction is referred to as an X2 direction (X2 side). Further, the direction orthogonal to the Z direction and the X direction is referred to as a Y direction (Y axis), one side along the Y direction is referred as a Y1 direction, and the other side along the Y direction is referred to as a Y2 direction.

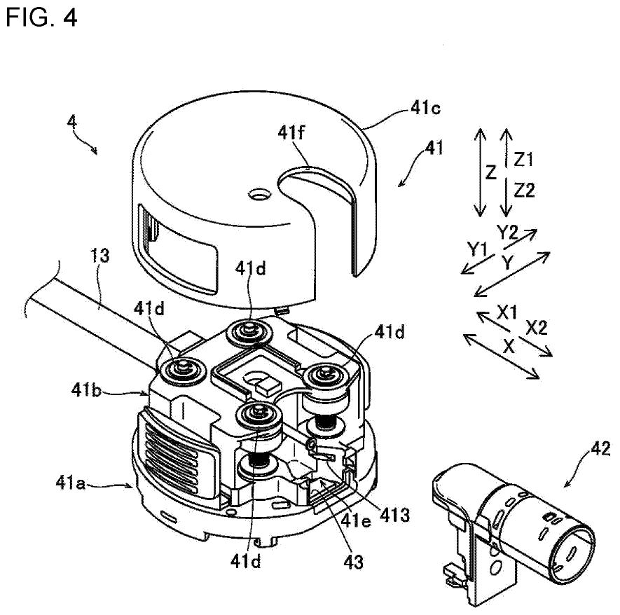

As illustrated in FIG. 4, the electrosurgical instrument 4 includes a housing 41, an electric connection part 42, and a circuit board 43.

(Housing)

The housing 41 constitutes a casing or housing part that accommodates therein a drive mechanism of the electrosurgical instrument 4. Specifically, the housing 41 includes a base 41a, a retaining member 41b, a lid portion 41c (or a cover), and a plurality (four) of driven members 41d.

The base 41a is formed with an adaptor attachment surface on the Z2 side of the base 41a. To the base 41a, the other end of the shaft 13 is connected. The base 41a includes a recess portion 41e in which the circuit board 43 is to be disposed. The recess portion 41e is recessed toward the Z2 side. The recess portion 41e is provided on an end portion of the base 41a on the Y2 side. The retaining member 41b holds the driven members 41d to be rotatable on the base 41a. The lid portion 41c covers the base 41a from the Z1 side. The lid portion 41c is detachably attached to the base 41a. The lid portion 41c is formed with a notch 41f or a cutout for disposing the electric connection part 42.

The driven members 41d are driven and rotated to drive the end effector 12 (see FIG. 3). Specifically, the end effector 12 is provided on the X1 side of the shaft 13. The end effector 12 is connected to the shaft 13 via a support member. The base 41a of the housing 41 is connected to the X2 side of the shaft 13. The driven members 41d are connected to the end effector 12 with wires passing through the shaft 13.

For example, the number of the driven members 41d provided is four. When one of the driven members 41d is rotated, the shaft 13 is rotated. When one or more of the other three driven members 41d are rotated, the end effector 12 is driven. The four driven members 41d are arranged with two rows (columns) in the X direction and two columns (rows) in the Y direction. The driven members 41d are provided in the housing 41.

### (Electric Connection Part)

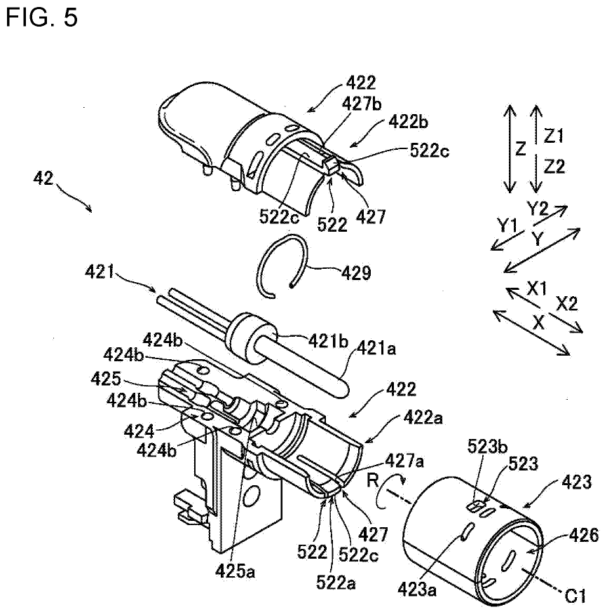

As illustrated in FIGS. 4 and 5, the electric connection part 42 according to an embodiment accommodates therein an electrode 421 to supply electric power to the end effector 12. The electric connection part 42 is electrically connected to the power supply 32, in such a manner that a cable 16 connected to the power supply 32 is connected with the electrode 421 of the electric connection part 42. Here, the electrode 421 is configured to supply the electric power supplied from the power supply 32 to the end effector 12 in order to generate heat in the end effector 12. The electric connection part 42 is configured to be divided into a plurality of members so that the electric connection part 42 can accommodate therein the electrode 421. Such a plurality of divided members of the electric connection part 42 are connected to each other to form one structure.

The electrosurgical instrument 4 includes: the housing 41 attached to the robot arm 6a; the shaft 13 having one end and the other end, wherein a side of the one end (X2 side) of the shaft 13 is connected to the housing 41; the end effector 12 provided on a side of the other end (X1 side) of the shaft 13; and the electric connection part 42 provided to the housing 41 to be electrically connected to the power supply 32. The electric connection part 42 includes: the electrode 421 to be electrically connected to the power supply 32; two divided members 422 that hold the electrode 421 therebetween; and a coupling member 423 that connects the two divided members 422.

According to this configuration, the electrode 421 can be held by the electric connection part 42 simply by connecting the plurality of divided members 422 to each other with the coupling member 423 in a state where the electrode 421 is sandwiched between the plurality of divided members 422. Therefore, the assembly work of the electrosurgical instrument 4 can be simplified.

### (Divided Members and Coupling Member)

Hereinafter, the plurality of divided members 422 and the coupling member 423 in the configuration of the electric connection part 42 are described.

The plurality of divided members 422 includes a first divided member 422a to be attached to the housing 41 and a second divided member 422b, wherein the first divided member 422a and the second divided member 422b sandwich the electrode 421 therebetween.

As a result, the number of the plurality of divided members 422 can be minimized (two), so that an increase in the number of parts of the electric connection part 42 can be suppressed, and the assembly work of the electrosurgical instrument 4 can be further simplified.

The first divided member 422a and the second divided member 422b are made of a resin material. The first divided member 422a and the second divided member 422b are divided along a dividing surface (dividing line). The dividing surface is a plane extending in the XY direction passing through the central portion of the electrode 421 in the Z direction.

The first divided member 422a of the electric connection part 42 is provided separately from the base 41a, the retaining member 41b, and the lid portion 41c. The first divided member 422a is engaged with the base 41a and thus is attached to the base 41a of the housing 41. The first divided member 422a is a portion of the electric connection part 42 on the Z2 side with respect to the dividing surface along which the electric connection part 42 is divided. The first divided member 422a supports the electrode 421 from the Z2 side. The second divided member 422b is provided separately from the first divided member 422a. The second divided member 422b is coupled with the first divided member 422a by the coupling member 423. The second divided member 422b is a portion of the electric connection part 42 on the Z1 side with respect to the dividing surface along which the electric connection part 42 is divided. The second divided member 422b functions as a cover member to cover the electrode 421 from the Z1 side.

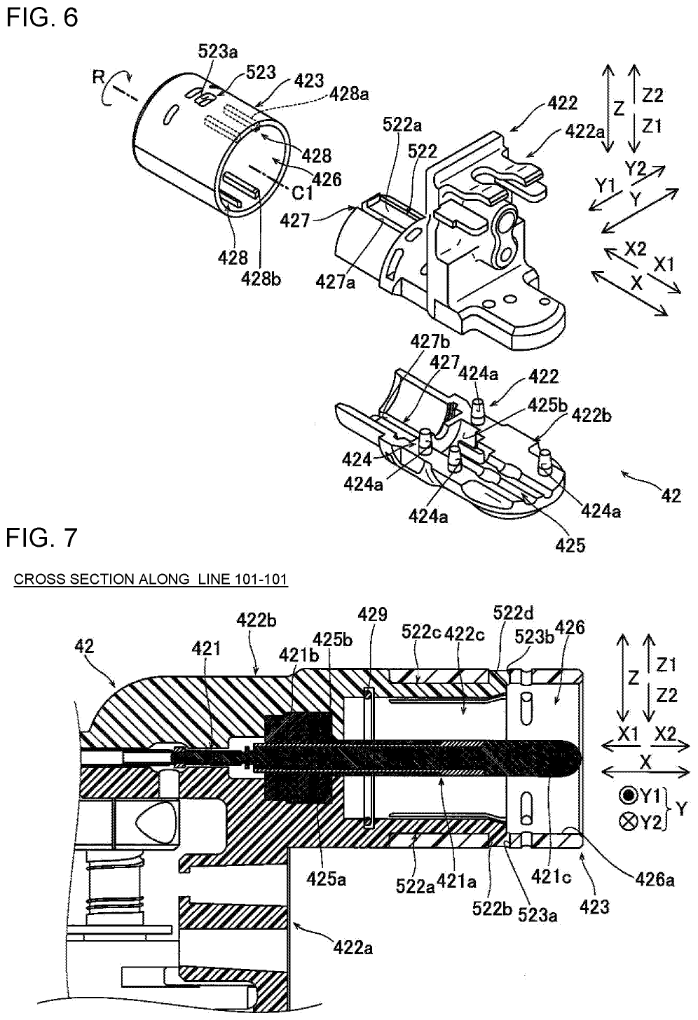

As illustrated in FIGS. 5 and 6, each of the first divided member 422a and the second divided member 422b includes a positioning part 424 for positioning the first divided member 422a and the second divided member 422b with respect to each other upon attaching the second divided member 422b to the first divided member 422a.

Accordingly, the positioning part 424 positions the first divided member 422a and the second divided member 422b with respect to each other, so that the first divided member 422a and the second divided member 422b can be coupled to each other by the coupling member 423 in a state where the first divided member 422a and the second divided member 422b are not displaced from each other. As a result, the first divided member 422a and the second divided member 422b can be appropriately coupled to each other with the coupling member 423 in the state where the positional relationship between the first divided member 422a and the second divided member 422b is accurately maintained.

The positioning part 424 is configured to position the second divided member 422b with respect to the first divided member 422a. Specifically, the positioning part 424 positions the first divided member 422a and the second divided member 422b with each other in the direction (the direction in which the XY plane extends) orthogonal to the direction (Z direction) in which the first divided member 422a and the second divided member 422b are adjacent to each other. At this time, the positioning part 424 is configured to position the first divided member 422a and the second divided member 422b with respect to each other and to temporarily hold the second divided member 422b on the first divided member 422a.

Specifically, the positioning part 424 of the second divided member 422b includes positioning pins 424a provided on the second divided member 422b and the positioning part 424 of the first divided member 422a includes pin insertion holes 424b provided in the first divided member 422a at the positions corresponding to the positioning pins 424a.

As a result, the first divided member 422a and the second divided member 422b can be positioned simply by inserting the positioning pins 424a into the pin insertion holes 424b. Therefore, the positioning of the first divided member 422a and the second divided member 422b can be realized with a simple structure and can be performed with a simple operation.

The positioning pin 424a has a circular column shape. A plurality (four) of positioning pins 424a are provided to the second divided member 422b. Two of the positioning pins 424a are arranged on the Y1 side and the other two are provided on the Y2 side on the second divided member 422b. Each of the positioning pins 424a is protruded from the dividing surface (the Z2 side surface) of the second divided member 422b toward the first divided member 422a. The pin insertion hole 424b has a shape corresponding to that of the positioning pin 424a. A plurality (four) of pin insertion holes 424b are provided to the first divided member 422a to correspond to the plurality (four) of positioning pins 424a. Two of the pin insertion holes 424b are arranged on the Y1 side and the other two are provided on the Y2 side on the first divided member 422a. Each of the pin insertion holes 424b is recessed from the dividing surface (the Z1 side surface) of the first divided member 422a toward the side (the Z2 side) away from the second divided member 422b.

The positioning pins 424a are inserted into the pin insertion holes 424b respectively upon attaching the second divided member 422b to the first divided member 422a. As a result, the plurality of positioning pins 424a and the plurality of pin insertion holes 424b are engaged with each other in the X direction and the Y direction. Thus, the relative movement of the first divided member 422a and the second divided member 422b in the extending direction of the dividing surface is restricted.

Each of the first divided member 422a and the second divided member 422b is formed with an accommodation recess 425 to accommodate the electrode 421. The accommodation recess 425 is formed by a plurality of recess portions having different depths which communicate each other. The electric connection part 42 accommodates therein the electrode 421 in a state where the movement of the electrode 421 is restricted.

As illustrated in FIG. 7, the electrode 421 is an electrode for a bipolar type electrosurgical instrument. The electrode 421 includes: an insertion portion 421a to be inserted into the connector 17 of the cable 16 to be attached to the connector 17; and an enlarged portion 421b larger than the insertion portion 421a in a direction orthogonal to the extending direction (X direction) of the insertion portion 421a. The first divided member 422a and the second divided member 422b respectively include a first recess portion 425a and a second recess portion 425b that accommodate the enlarged portion 421b and that regulate the movement of the electrode 421 in the direction (X2 direction or removal direction) opposite to the attachment direction (X1 direction) of the connector 17 to the insertion portion 421a.

Therefore, the movement of the electrode 421 in the direction (the X2 direction, or the removal direction) opposite to the attachment direction of the connector 17 to the insertion portion 421a can be restricted, by means of the first recess portion 425a, the second recess portion 425b, and the enlarged portion 421b provided in the electrode 421 for the bipolar type electrosurgical instrument. According to this configuration, by restricting the movement of the electrode 421 by the first recess portion 425a and the second recess portion 425b, it is possible to prevent the electrode 421 from moving in the direction in which the connector 17 is pulled out from the electrode 421 even if the connector 17 is repeatedly inserted and removed from the electrode 421. Therefore, when the connector 17 is pulled out from the electrode 421, the electrode 421 is prevented from being pulled out together with the connector 17, and the holding state of the electrode 421 by the plurality of divided members 422 can be maintained.

The first recess portion 425a and the second recess portion 425b are parts of the accommodation recess 425. Specifically, the first recess portion 425a and the second recess portion 425b are portions of the accommodation recess 425 in which the enlarged portion 421b is housed. The first recess portion 425a is recessed from the dividing surface (the Z1 side surface) of the first divided member 422a toward the side (the Z2 side) away from the second divided member 422b. The first recess portion 425a has a shape that corresponds to a Z2 side portion of the enlarged portion 421b. The second recess portion 425b is recessed from the dividing surface (the Z2 side surface) of the second divided member 422b toward the side (the Z1 side) away from the first divided member 422a. The second recess portion 425b has a shape that corresponds to a Z1 side portion of the enlarged portion 421b.

As illustrated in FIGS. 6 and 7, the coupling member 423 is inserted in a state where the first divided member 422a and the second divided member 422b are pressed, so as to couple the first divided member 422a and the second divided member 422b to each other. That is, in order to ensure the insulating performance, the coupling member 423 presses the first divided member 422a and the second divided member 422b against each other toward the dividing surface so as not to form a gap between the first divided member 422a and the second divided member 422b. In this way, the first divided member 422a and the second divided member 422b are fastened in the Z direction by the coupling member 423.

Specifically, the coupling member 423 has a tubular shape formed with an internal space (hollow space) 426 into which a part of the first divided member 422a and the second divided member 422b is inserted. The first divided member 422a and the second divided member 422b are connected in a state where the first divided member 422a and the second divided member 422b are inserted into the internal space 426 of the coupling member 423.

As a result, the first divided member 422a and the second divided member 422b can be coupled by the coupling member 423 having a simple structure, so that it is possible to prevent the structure of the electric connection part 42 from becoming complicated. Further, since the first divided member 422a and the second divided member 422b are inserted into the internal space 426 of the coupling member 423, the coupling member 423 can tighten the first divided member 422a and the second divided member 422b from the outside over the entire circumference. Accordingly, it is possible to prevent the first divided member 422a and the second divided member 422b from being separated from each other.

The coupling member 423 has a cylindrical tubular shape having an internal space 426 penetrating therethrough in the X direction. The portions of the first divided member 422a and the second divided member 422b on the X2 side are inserted into the internal space 426 of the coupling member 423 in the state where the first divided member 422a and the second divided member 422b are pressed against each other. Here, in the direction orthogonal to the X direction, the diameter of the internal space 426 is smaller than the diameter of the cylindrical portion formed by the portions of the first divided member 422a and the second divided member 422b on the X2 side. The coupling member 423 and the cylindrical portion formed by the X2 side portions of the first divided member 422a and the second divided member 422b have a dimensional relationship of a tight fit.

The coupling member 423 covers a portion 421c of the electrode 421 that is protruded from the first divided member 422a and the second divided member 422b.

According to this configuration, the portion 421c of the electrode 421 that is protruded from the divided members (the first divided member 422a and second divided member 422b) can be protected, so that it is possible to prevent an excessive load from being directly applied to the electrode 421.

Here, the portion 421c of the electrode 421 that is protruded from the first divided member 422a and the second divided member 422b is a portion of the insertion portion 421a on the X2 side. That is, the coupling member 423 surrounds the portion of the insertion portion 421a on the X2 side in the direction orthogonal to the X direction. The end portion of the coupling member 423 on the X2 side is arranged on the X2 side with respect to the end portion of the insertion portion 421a of the electrode 421 on the X2 side. In this way, the coupling member 423 has a function as a protective member for protecting the electrode 421.

The electrode 421 includes the insertion portion 421a to which the connector 17 of the cable 16 for electrically connecting the power supply 32 and the electrode 421 is inserted and thus attached. The insertion portion 421a is provided over a first space 422c defined by the first divided member 422a and the second divided member 422b and a second space 426a formed inside the coupling member 423.

According to this configuration, by arranging the insertion portion 421a in the space extending over the first space 422c and the second space 426a, the circumference of the insertion portion 421a is surrounded by the first divided member 422a, the second divided member 422b, and the coupling member 423. Since it can be enclosed, the insertion portion 421a can be protected by the first divided member 422a, the second divided member 422b, and the coupling member 423.

As illustrated in FIG. 7, the first space 422c is a space surrounded by the first divided member 422a and the second divided member 422b in the direction orthogonal to the X direction. Specifically, the first space 422c is a space surrounded by a portion of the first divided member 422a on the X2 side and a portion of the second divided member 422b on the X2 side. That is, the Z1 side portion of the first space 422c is surrounded by the inner circumferential surface of the X2 side portion of the first divided member 422a. The Z2 side portion of the first space 422c is surrounded by the inner circumferential surface of the X2 side portion of the second divided member 422b.

The second space 426a is a part (an X2 side portion) of the internal space 426 of the coupling member 423. That is, the second space 426a is a space surrounded by the coupling member 423 in the direction orthogonal to the X direction. Specifically, the second space 426a is surrounded by the inner circumferential surface of the X2 side portion of the coupling member 423.

As illustrated in FIGS. 5 and 6, the coupling member 423 is configured to be guided to a predetermined position when the first divided member 422a and the second divided member 422b with being pressed against each other are inserted into the coupling member 423. Further, when the first divided member 422a and the second divided member 422b with being pressed against each other are inserted into the coupling member 423, the coupling member 423 is moved in the insertion direction (X1 direction) of the coupling member 423 in a state where the coupling member 423 is positioned so as not to rotate along the R direction about the axis C1.

The first divided member 422a and the second divided member 422b include a coupling member guide portion 427 that guides the coupling member 423 along the insertion direction (X1 direction) in which the coupling member 423 is inserted to the first divided member 422a and the second divided member 422b and that regulates the rotation of the coupling member 423 in the circumferential direction (R direction) about the axis C1 parallel to the insertion direction (X1 direction). The coupling member 423 includes a second guide portion 428 that is guided by the coupling member guide portion 427 serving as a first guide portion, such that the rotation of the coupling member 423 is restricted.

With this configuration, the first guide portion 427 and the second guide portion 428 can suppress the relative positional deviation of the coupling member 423 with respect to the first divided member 422a and the second divided member 422b in the R direction, and also can guide the coupling member 423 to easily move along the X1 direction. As a result, the work of assembling the coupling member 423 to the first divided member 422a and the second divided member 422b can be performed accurately and easily.

Specifically, the first guide portion 427 is composed of a guide groove portion extending along the X1 direction. The guide groove portion penetrates the first divided member 422a and the second divided member 422b in the Z direction. The guide groove portion includes a pair of first guide groove portions 427a formed in the Z2 side portion of the first divided member 422a and a pair of second guide groove portions 427b formed in the Z1 side portion of the second divided member 422b.

Further, the second guide portion 428 is composed of protruded portions to be inserted into the guide groove portions from the X2 side (the side opposite to the insertion direction). Each of the protruded portions is protruded inwardly from the inner circumferential surface of the coupling member 423. The protruded portions includes a pair of first protruded portions 428a arranged on the Z2 side corresponding to the pair of first guide groove portions 427a and a pair of second protruded portions 428b arranged on the Z1 side corresponding to the pair of second guide groove portions 427b.

Here, the pair of first protruded portions 428a and the pair of first guide groove portions 427a are engaged with each other in the R direction, and the pair of second protruded portions 428b and the pair of second guide groove portions 427b are engaged with each other in the R direction.

The coupling member 423 is attached to the first divided member 422a and the second divided member 422b in the state where the first divided member 422a and the second divided member 422b are pressed inwardly and inserted into the coupling member 423. Here, the coupling member 423 is attached to the first divided member 422a and the second divided member 422b, such that the movement of the coupling member 423 in the X2 direction is restricted.

As illustrated in FIGS. 5 to 7, the coupling member 423 includes an engagement portion 523. Each of the first divided member 422a and the second divided member 422b includes an elastically deformable engagement portion 522 to be engaged with the engagement portion 523 of the coupling member 423 in the state where the first divided member 422a and the second divided member 422b are coupled.

Since the coupling member 423 can be attached to the first dividing member 422a and the second dividing member 422b without using an adhesive or screws, the work of attaching the coupling member 423 to the first dividing member 422a and the second dividing member 422b can be easily performed.

The engagement portion 523 of the coupling member 423 is formed of engagement holes penetrating through the coupling member 423 in the Z direction. The engagement holes include a first engagement hole 523a provided in the Z2 side portion of the coupling member 423 and a second engagement hole 523b provided in the Z1 side portion of the coupling member 423. The engagement portions 522 are formed of snap-fits having claws to be inserted into the engagement holes. The snap-fits include a first snap-fit portion 522a having a first claw 522b (see FIG. 7) provided in the first divided member 422a corresponding to the first engagement hole 523a and a second snap-fit portion 522c having a second claw 522d (see FIG. 7) provided in the second divided member 422b corresponding to the second engagement hole 523b. The first snap-fit portion 522a and the second snap-fit portion 522c extend in the X direction.

A pair of first guide groove portions 427a are formed on both sides of the first snap-fit portion 522a in the R direction. With this, the first snap-fit portion 522a is configured to be elastically deformable in the Z direction. A pair of second guide groove portions 427b are formed on both sides of the second snap-fit portion 522c in the R direction. With this, the second snap-fit portion 522c is configured to be elastically deformable in the Z direction.

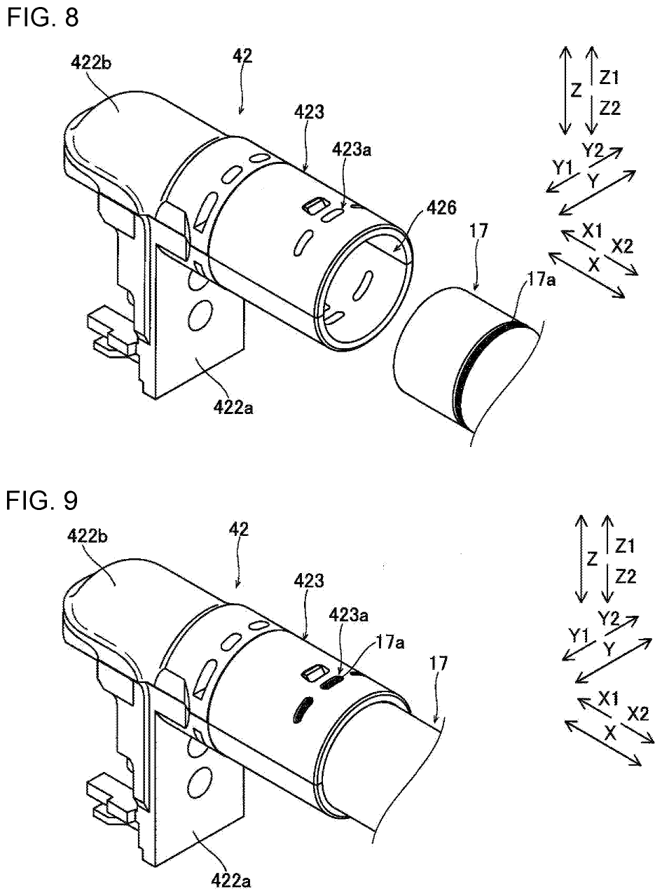

As illustrated in FIGS. 8 and 9, the coupling member 423 includes a configuration which allows the operator to recognize whether or not the connection with the connector 17 is completed.

Specifically, the coupling member 423 includes viewing holes 423a (or visual confirmation holes) that make a mark portion 17a of the connector 17 of the cable 16 visible in the state where the connector 17 is inserted in the coupling member 423 and connected to the electrode 421. The viewing holes 423a are examples of a hole that makes the mark portion visible.

As a result, the operator can confirm the insertion position of the connector 17 using the mark portion 17a and the viewing holes 423a, so that the connector 17 can be reliably connected to the electrode 421.

The mark portion 17a of the connector 17 is formed in an annular shape along the R direction around the axis C1. The mark portion 17a of the connector 17 is fitted into a fitting groove formed on the outer circumferential surface of the connector 17 along the R direction around the axis C1. The mark portion 17a has a color different from that of other parts of the connector 17. The viewing holes 423a penetrate the coupling member 423 in the Z direction. A plurality (six) of viewing holes 423a are arranged in the R direction. Among the six viewing holes 423a, three viewing holes 423a are provided in the first divided member 422a and the other three viewing holes 423a are provided in the second divided member 422b.

As illustrated in FIG. 8, in the state before the connector 17 is inserted into the internal space 426 of the coupling member 423, the mark portion 17a cannot be visually recognized through the viewing holes 423a. Then, when the connector 17 is pushed into the internal space 426 of the coupling member 423 so that the insertion portion 421a of the electrode 421 comes to the insertion completion position in the connector 17, the mark portion 17a is visually recognized as illustrated in FIG. 9. At the insertion completion position, the connector 17 is engaged with an engagement member 429 (see FIG. 7) attached to the inner circumference of the first divided member 422a and the second divided member 422b. The engagement member 429 is composed of a spring that can be elastically deformable in a direction orthogonal to the X direction. Specifically, the engagement member 429 is composed of a ring-shaped (C-shaped) wire spring.

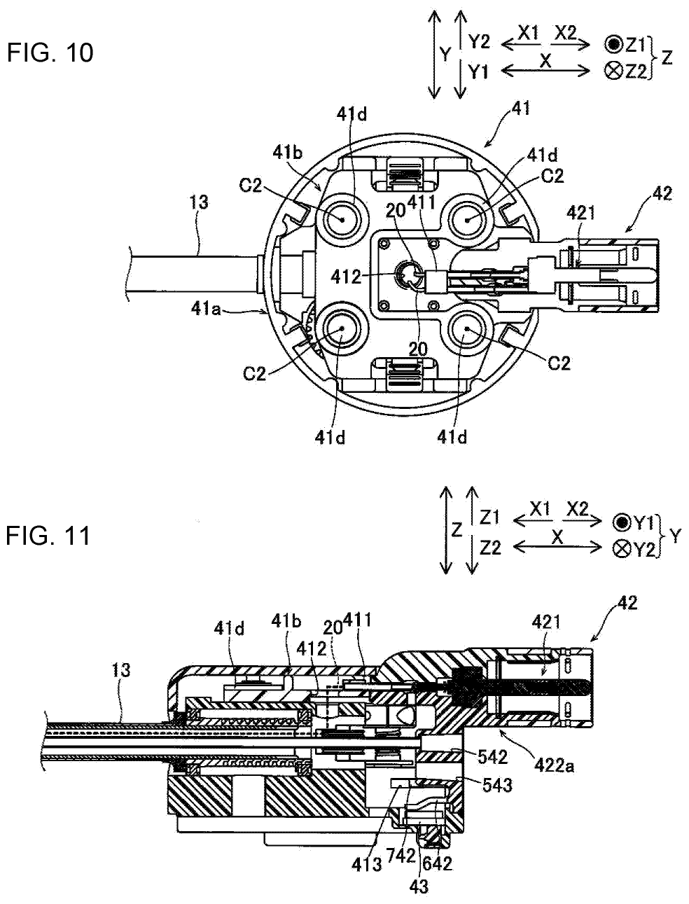

As illustrated in FIGS. 10 and 11, the housing 41 is provided with the driven members 41d that are rotationally driven by driving parts provided on the robot arm 6a, and a retaining member 41b that rotatably holds the driven members 41d. The retaining member 41b includes an electrical conductor guide portion 411 that guides an electrical conductor (lead wire) 20 for transmitting electrical energy from the electrode 421 to the end effector 12 (see FIG. 3).

As a result, the wiring of the electrical conductor 20 can be easily performed, so that the workability of the operator during the connection work can be improved.

The retaining member 41b includes a through hole 412 which penetrates in the extending direction of the rotation axis C2 of the driven members 41d and through which the electrical conductor 20 guided by the electrical conductor guide portion 411 is passed to introduce the electrical conductor 20 into the shaft 13.

As a result, the electrical conductor 20 can be wired from the electrode 421 toward the end effector 12 side merely by passing the electrical conductor 20 through the through hole 412, so that the wiring of the electrical conductor 20 can be performed more easily.

The electric connection part 42 is configured to constitute the housing 41 together with the base 41a and the lid portion 41c. The electric connection part 42 includes a first cleaning liquid supply port 542 and a second cleaning liquid supply port 543 (flash ports), a pair of press portions 642, and a pair of engagement projections 742. The first cleaning liquid supply port 542 and the second cleaning liquid supply port 543 are examples of a cleaning liquid supply port.

The first divided member 422a is formed with the first cleaning liquid supply port 542 and and the second cleaning liquid supply port 543 to supply a cleaning liquid.

As a result, the electrosurgical instrument 4 can be washed by using the first cleaning liquid supply port 542 and the second cleaning liquid supply port 543, so that the electrosurgical instrument 4 can be kept clean.

The first cleaning liquid supply port 542 and the second cleaning liquid supply port 543 are provided to supply a cleaning liquid (such as water). The first cleaning liquid supply port 542 and the second cleaning liquid supply port 543 penetrate the first divided member 422a in the X direction. The first cleaning liquid supply port 542 and the second cleaning liquid supply port 543 are arranged side by side in the Z direction. The first cleaning liquid supply port 542 is provided on the Z1 side to supply the cleaning liquid to the inside of the shaft 13. The second cleaning liquid supply port 543 is provided on the Z2 side to supply the cleaning liquid to the inside of the housing 41.

The housing 41 includes the base 41a to which the other end of the shaft 13 is connected. The electrosurgical instrument 4 includes a circuit board 43 provided on the base 41a. The first divided member 422a includes press portions 642 that press the circuit board 43 to the base 41a to hold the circuit board 43.

With this configuration, since the circuit board 43 is held on the base 41a by the pair of pressing portions 642, the circuit board 43 can be easily attached to the base 41a, unlike the case where the circuit board 43 is attached to the base 41a by, for example, a screw or the like.

The pair of press portions 642 are configured to press the circuit board 43 to the base 41a so as to hold the circuit board 43. Specifically, the pair of press portions 642 are configured to be elastically deformed (resiliently deformed) to press the circuit board 43 to the base 41a so as to hold the circuit board 43. Specifically, the pair of press portions 642 are made of an insulating resin material. The pair of press portions 642 are provided to extend in the X direction. The pair of press portions 642 extend substantially in parallel to each other. The pair of press portions 642 are opposed to each other in the Y direction. The pair of press portions 642 press both end portions, in the Y direction, of the circuit board 43, so as to hold the circuit board 43.

The circuit board 43 is a memory board. The circuit board 43 serving as the memory board can be easily attached to the base 41a by means of the pair of press portions 642. In the memory of the circuit board 43, information about the electrosurgical instrument 4 such as the type of the electrosurgical instrument 4 and the number of times the electrosurgical instrument 4 has been used are stored, for example.

The circuit board 43 is provided with a circuit portion (not illustrated) and an electrode array (not illustrated). The circuit portion is provided on a surface of the circuit board 43 on the Z1 side. The circuit portion includes, for example, electronic components such as ROM (Read Only Memory) and/or the like. The electrode array is provided at a surface of the circuit board 43 on the Z2 side. The electrode array is electrically connected to an electrode array of the robot arm 6a through an electrode array of the adaptor 11. Accordingly, the information stored in the circuit board 43 can be grasped on the robot arm 6a side.

The first divided member 442a is configured to be attached to the base 41a by being slid with respect to the base 41a.

As a result, the first divided member 422a can be easily attached to the base 41a, so that the assembling work of the electrosurgical instrument 4 can be simplified.

Specifically, a pair of engagement projections 742 is provided to the first divided member 422a. The pair of engagement projections 742 is configured to be engaged with the base 41a. The pair of engagement projections 742 is configured to be engaged with a pair of engagement recesses 413 (see FIG. 4) of the base 41a, respectively. With the pair of engagement projections 742 being engaged with the pair of engagement recesses 413, the first divided member 442a is connected to the base 41a so as not to come off the base 41a in the Z direction. The pair of engagement projections 742 extend in the X direction with being parallel to each other. The pair of engagement projections 742 are opposed to each other in the Y direction. The pair of engagement projections 742 are provided on the Z1 side with respect to the pair of press portions 642.

### [Modification]

It should be understood that one or more embodiments described above are illustrated by way of example in every respect and not limit the disclosure. The scope of the invention is indicated by claims and includes equivalents to the claims and all alterations (modification) within the same.

For example, in one or more embodiments described above, the case has been described in which the first divided member 422a and the second divided member 422b are provided separately from the base 41a of the housing 41. However, the invention is not limited to this. For example, in the invention, a first divided member may be integrally formed with a base.

In one or more embodiments described above, the case has been described in which the divided members 422 includes two divided members 422a and 422b. However, the invention is not limited to this. In the invention, divided members may include three or more divided members.

In one or more embodiments described above, the case has been described in which the first divided member 422a includes the pin insertion holes 424b and the second divided member 422b includes the positioning pins 424a. However, the invention is not limited to this. In the invention, a first divided member may include a positioning pin(s) and a second divided member may include a pin insertion hole(s).

In one or more embodiments described above, the case has been described in which the first divided member 422a and the second divided member 422b are positioned by the positioning pins 424a and the pin insertion holes 424b. However, the invention is not limited to this. In the invention, a first divided member and a second divided member may be engaged with each other without being positioned.

In one or more embodiments described above, the case has been described in which the coupling member 423 has the cylindrical tubular shape having an internal space 426 penetrating in the X direction (the insertion direction). However, the invention is not limited to this. In the invention, a coupling member may have a polygonal tubular shape having an internal space or the like.

In one or more embodiments described above, the case has been described in which the retaining member 41b includes the electrical conductor guide portion 411. However, the invention is not limited to this. In the invention, a retaining member may not include an electrical conductor guide portion.

In one or more embodiments described above, the case has been described in which the electrosurgical instrument includes the pair of press portions 642. However, the invention is not limited to this. In the invention, a press portion may be provided to a base or a lid portion of a housing.

In one or more embodiments described above, the case has been described in which the pair (two) of press portions 642 are provided. However, the invention is not limited to this. In the invention, the number of a press portion(s) may be one or more than two.

In one or more embodiments described above, the case has been described in which the circuit board is the memory circuit. However, the invention is not limited to this. In the invention, a circuit board may be a circuit board other than a memory board.

In one or more embodiments described above, the case has been described in which the coupling member 423 covers the portion 421c of the electrode 421 that protrudes from the first divided member 422a and the second divided member 422b. However, the invention is not limited to this. In the invention, a coupling member may not cover a portion of an electrode that protrudes from first and second divided members.

In one or more embodiments described above, the case has been described in which the first divided member 422a and the second divided member 422b include the first guide portion 427, and the coupling member 423 includes the second guide portion 428. However, the invention is not limited to this. In the invention, first and second divided members may not include a first guide portion, and a coupling member may not include a second guide portion.

In one or more embodiments described above, the case has been described in which both of the first divided member 422a and the second divided member 422b include the first guide portion 427. However, the invention is not limited to this. In the invention, a first or second divided member may include a first guide portion.

In one or more embodiments described above, the case has been described in which the coupling member 423 includes the hole through which the mark portion 17a of the connector 17 can be visually recognized. However, the invention is not limited to this. In the invention, a coupling member may have a structure that makes a sound when a connector is inserted into the coupling member.

1. An electrosurgical instrument comprising:
a housing to be attached to a robot arm;
a shaft including one end and the other end, wherein a side of the one end of the shaft is connected to the housing;
an end effector provided on a side of the other end of the shaft; and
an electric connection part provided to the housing to be electrically connected to a power supply, wherein
the electric connection part includes:
an electrode to be electrically connected to the power supply;
a plurality of divided members configured to hold the electrode therebetween; and
a coupling member that couples the plurality of divided members.

2. The electrosurgical instrument according to claim 1, wherein
the plurality of divided members includes:
a first divided member attached to the housing; and
a second divided member, wherein the first divided member and the second divided member hold the electrode therebetween.

3. The electrosurgical instrument according to claim 2, wherein
each of the first divided member and the second divided member includes a positioning part that positions the first divided member and the second divided member with respect to each other.

4. The electrosurgical instrument according to claim 3, wherein
the positioning part includes:
a positioning pin provided to one of the first divided member and the second divided member; and
a pin insertion hole provided to the other of the first divided member and the second divided member so as to correspond to the positioning pin.

5. The electrosurgical instrument according to claim 2, wherein
the coupling member has a tubular shape into which at least a part of the first divided member and the second divided member is inserted.

6. The electrosurgical instrument according to claim 1, wherein
the housing is provided with:
a driven member configured to be driven to rotate by a driving part provided to the robot arm; and
a retaining member that holds the driven member to be rotatable, wherein
the retaining member includes an electrical conductor guide portion that guides an electrical conductor for transmitting electrical energy from the electrode to the end effector.

7. The electrosurgical instrument according to claim 6, wherein
the retaining member further includes a through hole which penetrates in an extending direction of a rotation axis of the driven member and through which the electrical conductor guided by the electrical conductor guide portion is passed to introduce the electrical conductor into the shaft in a tubular shape.

8. The electrosurgical instrument according to claim 2, wherein
the coupling member includes an engagement portion, and
each of the first divided member and the second divided member includes an elastically deformable engagement portion to be engaged with the engagement portion of the coupling member.

9. The electrosurgical instrument according to claim 2, wherein
the first divided member is provided with a cleaning liquid supply port to supply a cleaning liquid.

10. The electrosurgical instrument according to claim 2, wherein
the housing includes a base to which the other end of the shaft is connected,
the electrosurgical instrument further comprises a circuit board provided on the base,
the first divided member includes a press portion that presses the circuit board to the base to hold the circuit board.

11. The electrosurgical instrument according to claim 10, wherein
the first divided member is configured to be attached to the base by a slide movement of the first divided member with respect to the base.

12. The electrosurgical instrument according to claim 10, wherein
the first divided member includes a pair of engagement projections, and
the base includes a pair of engagement recesses to which the pair of engagement projections is to be engaged.

13. The electrosurgical instrument according to claim 2, wherein
the coupling member covers a portion of the electrode that protrudes from the first divided member and the second divided member.

14. The electrosurgical instrument according to claim 2, wherein
the electrode includes an insertion portion to which a connector of a cable for electrically connecting the power supply and the electrode is inserted and thus attached, and
the insertion portion extends over a first space defined by the first divided member and the second divided member and a second space formed inside the coupling member.

15. The electrosurgical instrument according to claim 14, wherein
the electrode is an electrode for a bipolar type electrosurgical instrument,
the electrode includes an enlarged portion larger than the insertion portion in a direction orthogonal to an extending direction of the insertion portion, and
the first divided member and the second divided member include recess portions that accommodate the enlarged portion of the electrode and regulate a movement of the electrode in a direction opposite to an attachment direction in which the connector is attached to the insertion portion.

16. The electrosurgical instrument according to claim 2, wherein
at least one of the first divided member and the second divided member includes a first guide portion that guides the coupling member along an insertion direction in which the coupling member is inserted to the first divided member and the second divided member and that restricts rotation of the coupling member in a circumferential direction about an axis parallel to the insertion direction, and
the coupling member includes a second guide portion that is guided by the first guide portion to restrict the rotation of the coupling member.

17. The electrosurgical instrument according to claim 1, wherein
the coupling member includes a hole that makes a mark portion provided on a connector of a cable for electrically connecting the power supply and the electrode visible in a state where the connector is connected to the electrode.

18. The electrosurgical instrument according to claim 1, wherein
the divided members include a C-shaped spring to be engaged with a connector of a cable for electrically connecting the power supply and the electrode.

19. An electrosurgical instrument comprising:
a base to be attached to a robot arm;
a lid portion that covers the base and includes a notch;
a shaft including one end and the other end, wherein a side of the one end of the shaft is connected to the base;
an end effector provided on a side of the other end of the shaft; and
an electric connecting part provided at the notch of the lid portion to be electrically connected to a power supply, wherein
the electric connection part includes:
an electrode to be electrically connected to the power supply;
a plurality of divided members configured to hold the electrode therebetween; and
a coupling member that couples the plurality of divided members.

20. A surgical system comprising:
a patient-side apparatus including robot arms to which an endoscope and an electrosurgical instrument are attached; and
an image processing apparatus including a power supply and configured to process an image captured by the endoscope, wherein
the electrosurgical instrument includes:
a housing that is attached to one of the robot arms;
a shaft including one end and the other end, wherein a side of the one end of the shaft is connected to the housing;
an end effector provided on a side of the other end of the shaft; and
an electric connection part provided to the housing and electrically connected to the power supply, wherein
the electric connection part includes:
an electrode to be electrically connected to the power supply via a cable;
a plurality of divided members configured to hold the electrode therebetween; and
a coupling member that couples the plurality of divided members.
